(19) United States
(12) Patent Application Publication
(10) Pub. No.: US 2004/0146983 A1
(43) Pub. Date: Jul. 29, 2004

(54) Enhanced production of Streptococcus mutans mutacin I and III

(76) Inventors: Page W. Caufield, New York, NY (US); Zengguo He, Brooklyn, NY (US)

Correspondence Address: KLAUBER & JACKSON, 411 HACKENSACK AVENUE, HACKENSACK, NJ, 07601

(21) Appl. No.: 10/672,784

(22) Filed: Sep. 26, 2003

Related U.S. Application Data
(60) Provisional application No. 60/413,807, filed on Sep. 26, 2002.

(51) Int. Cl. C12P 21/02; C12N 1/21

(52) U.S. Cl. 435/69.3; 435/252.3

(57) ABSTRACT

Methods for producing a mutacin, by growing a mutacin-producing cell in a liquid medium under conditions in which mutacin is produced and isolating mutacin from the liquid medium. Methods are also provided for isolating and purifying mutacin I/III from fermentation broths of Streptococcus mutans strains CH43 and UA787 to homogeneity. Fermentation are conducted in anaerobic bioreactors under anaerobic conditions at a cultivation temperature of about 35-42° C., with agitation rate of between 50-250 rpm.

### CROSS REFERENCE TO RELATED APPLICATIONS

This non-provisional patent application claims, in accordance with 35 USC § 119 (e), the priority date of U.S. Provisional Patent Application No. 60/413,807, filed Sep. 26, 2002, which is hereby incorporated by reference in its entirety.

### STATEMENT OF GOVERNMENT SUPPORT

The research leading to the present invention was supported in part by NIH grant RO1 DE09082. The government may have certain rights in the present invention.

### FIELD OF THE INVENTION

This invention relates to a production method for achieving high yields of mutacins I and III.

### BACKGROUND OF THE INVENTION

A heterogeneous group of peptide antibiotics called mutacins are produced by Streptococcus mutans (Hamada et al. (1975) Arch. Oral Biol. 20:641-648). Mutacin I, II, III belong to the lantibiotic class of bacteriocins, as they contain post-translational modified amino acids called lanthionines, while mutacin IV is a nonlantibiotic (de Vos et al. (1995) Mol. Microbiol. 17:427-437; Sahl et al. (1998) Ann. Rev. Microbiol. 52:41-79).

Mutacins exhibit both unique and overlapping antibacterial spectra, and are most active against gram-positive bacteria, particularly against members of the same or closely related species. Among them, mutacin I and III are the most effective mutacins. The mature mutacin I has 24 amino acids, whereas mutacin III has 22 amino acids. The peptide composition, secondary and tertiary structures are mediated by thioether bridging which likely contributes to the antibacterial properties of the mutacin, as well as other lantibiotics. Because mutacins interact with cell membranes, they possess both hydrophilic and hydrophobic domains. The amphipathic nature of mutacins, along with their relatively extensive post-translational modifications and tightly controlled regulation, contribute to the difficulty in isolating mutacins from growth media.

Experimental work shows that mutacin production is dependent on media and culture conditions. A medium suitable for production of one mutacin may not be appropriate for another. A liquid chemically defined medium supplemented with yeast extract and Trpticase soy was optimized for mutacin II production, but this medium failed to produce mutacin I and III when production was attempted. (Novák et al. (1994) J. Bacteriol. 176:44316-4320).

Due to the potential therapeutic value of mutacin use in the treatment of human bacterial infections, production methods capable of achieving high yields of mutacins are desirable.

### SUMMARY OF THE INVENTION

Although mutacin III has been purified from Petri dish culture of Streptococcus mutans UA 787, efforts to obtain large quantity of mutacin III have been frustrated by the lack of any suitable method for mutacin III production in a liquid culture. Recently, a liquid medium for mutacin I and III production in a level flask has been found. Using this medium, mutacin III yield amounted to 32,000 AU/ml in a spinner mini-bioreactor (New Brunswick Scientific Co.)

Although mutacins can be produced easily on solid medium culture in small quantities, it is difficult to acquire mutacin in liquid culture, even when the same medium is applied. For example, mutacin I and III could be produced in Todd Hewitt (TH, DIFCO Laboratories, Detroit, Mich.) plates, but none of them was detectable in liquid TH broth. An initial attempt to obtain mutacin I and mutacin III in large scale was largely delayed due to the low productivity of these mutacins in liquid culture (Qi et al. (1999a) Appl. Environ. Microbiol. 65:652-658; Qi et al (1999b) Appl. Environ. Microbiol. 65:3880-3887).

The present invention is based in part on the discovery of a liquid medium for mutacin I/III production and production methods which produce high yields of mutacin I/III, approximately 16,000 AU/ml and 32,000 AU/ml, respectively. These high yields are obtained using a combination of the liquid media used for mutacin I/III fermentation with a spinner mini-bioreactor (New Brunswick Scientific Co.). The mutacin I/III production may be further optimized to yield up to 50,000 AU/l under specific conditions, such as by fermentation in a software controlled fermentor (Bioflo™, New Brunswick Scientific Co.)

Accordingly, in a first aspect, the invention features a method for producing a mutacin, comprising growing a mutacin-producing cell in a liquid medium, wherein the liquid medium comprises yeast extract, peptone, a carbon source, and salts, under conditions in which mutacin is produced; and isolating mutacin from the liquid medium. In a more specific embodiment, the liquid medium comprises 10-60 g yeast extract, 10-60 g peptone, and 10-50 g of a carbon source (e.g., glucose, fructose, lactose and/or sucrose) per liter. In a more specific embodiment, the liquid medium comprises 30 g yeast extract, 20 g peptone, and 20 g sucrose per liter. In one embodiment, the salt content comprises K₂HPO₄, NaCl, MgSO₄. In a more specific embodiment, the liquid medium comprises 0.5-10 g K₂HPO₄, 1-15 g NaCl, and 0.1-20 g MgSO₄.7 H₂O per liter. In a more specific embodiment, the liquid medium comprises 2 g K₂HPO₄, 2 g NaCl, and 1 g MgSO₄.7 H₂O per liter. In a more specific embodiment, the liquid medium comprises 30 g yeast extract, 20 g peptone, 20 g sucrose 2 g K₂HPO₄, 2 g NaCl, and 1 g MgSO_{4—}7 H₂O per liter. The peptone may be, for example, Bacto™ peptone (DIFCO Laboratories, Detroit, Mich.).

In specific embodiments, the mutacin produced is mutacin I and/or mutacin III. A mutacin-producing cell is a Streptococcus mutans cell. More specifically, the Streptococcus mutans cell is Streptococcus mutans UA787 and/or Streptococcus mutans CH43.

In one embodiment, the conditions under which mutacin is produced are aerobic liquid culture conditions under which fermentation proceeds. More specifically, mutacin fermentation may be conducted in a bioreactor at a cultivation temperature of 35-42° C. In a more specific embodiment, the fermentation is conducted in a bioreactor at a cultivation temperature of 37° C. with a agitation rate of 150 rpm and an initial pH between 3.0-7.2. In a more specific embodiment, the fermentation is conducted in a bioreactor at a cultivation temperature of 37° C. and an initial agitation rate between 50-250 rpm. In a more specific embodiment, the initial pH is 5.6. In a more specific embodiment, the initial pH is 5.6, and the pH is maintained at 5.6 throughout the whole fermentation process.

In one embodiment, mutacin is isolated from the liquid culture after fermentation by removal of cells to obtain a cell-free liquid culture. In a more specific embodiment, the fermentation broth is centrifuged to obtain a cell-free fraction, extracted with chloroform, and the emulsion layer formed between the chloroform and aqueous phases centrifuged to isolate a pellet comprising mutacin. In a more specific embodiment, the fermentation broth is centrifuged to obtain a cell-free supernatant, extracted by adsorption to a column of hydrophobic resin, such as for example XAD-16, to retain mutacin, and further desorpting mutacinI/III by organic solvents. It will be understood that other types of hydrophobic resins, such as XAD-2, XAD-4, XAD-7, XAD-11, XAD-1180, XAD-2000 may also be used for the hydrophobic chromatography, although XAD-16 has been the most efficacious hydrophobic resin used in the present invention. Desorption of mutacin I//III is carried out by ethanol or other suitable organic solvents (non-limiting examples thereof includes isopropanol, methanol acetonitrile and the like). Pooled mutacin-containing fractions are resulted in a crude mutacin powder extracts. The crude powder was dissolved by 6 M urea and purified by a reverse-phase 30 cm SOURCE 15RPC custom column, using a fragmented gradient of A (0.1% trifluoroacetic acid (TFA)) and B (0.085% TFA in 60% acetonitrile). Elution was carried out with a fragmented gradient of solvent A and B using an AKTA Purifier (Amersham Pharmacia Biotech,Piscataway, N.J.). In a more specific embodiment, generated mutacin is further purified by to homogeneity by a 15 cm SOURCE 5RPC reverse-phase column, using a fragmented gradient of A (0.1% trifluoroacetic acid [TFA]) and B (0.085% TFA in 60% acetonitrile).

In a second aspect, the invention features a method for producing mutacin I and/or mutacin III, comprising growing a mutacin-producing cell in a liquid medium under conditions in which mutacin is produced, wherein the liquid medium comprises 30 g yeast extract, 20 g peptone, 20 g sucrose, and salts; and isolating mutacin I and/or III from the liquid medium. In one embodiment, the salt content comprises K₂HPO₄, NaCl, MgSO₄. In a more specific embodiment, the liquid medium comprises 2 g K₂HPO₄, 2 g NaCl, and 1 g MgSO₄.7 H₂O per liter. In a more specific embodiment, the liquid medium comprises 30 g yeast extract, 20 g peptone, 20 g sucrose 2 g K₂HPO₄, 2 g NaCl, and 1 g MgSO₄.7 H₂O per liter. The peptone may be, for example, Bacto™ peptone (DIFCO Laboratories, Detroit, Mich.). A mutacin-producing cell is a Streptococcus mutans cell. More specifically, the Streptococcus mutans cell is Streptococcus mutans UA787 and/or Streptococcus mutans CH43. In more specific embodiments, the mutacin-producing cell is fermented in a bioreactor at a cultivation temperature of 37° C. In a more specific embodiment, the fermentation is conducted in a bioreactor at a cultivation temperature of 37° C. with an agitation rate of 150 rpm in the absence of supplied air. In one embodiment, mutacin I and/or III is isolated from the liquid culture after fermentation by removal of cells to obtain a cell-free liquid culture. In a more specific embodiment, the fermentation broth is centrifuged to obtain a cell-free fraction, extracted by adsorption to a column of hydrophobic resin to retain mutacin, and further desorpting mutacin I/III by organic solvents, such as for example, ethanol.

In a third aspect, the invention features a method for producing mutacin I and/or mutacin III, comprising (a) growing a Streptococcus mutans cell in a liquid medium under fermentation conditions in which mutacin I and/or III is produced, wherein the liquid medium comprises 30 g yeast extract, 20 g peptone, 20 g sucrose 2 g K₂HPO₄, 2 g NaCl, and 1 g MgSO₄.7 H₂O per liter, and fermentation is conducted in a bioreactor at a cultivation temperature of 37° C. with a agitation rate of 150 rpm in the absence of supplied air; and (b) isolating mutacin I and/or III from the liquid culture, wherein a yield of about 16,000 AU/l of mutacin I and/or 32,000 AU/l of mutacin III are produced. In specific embodiments, the peptone is Bacto peptone. In another embodiment, the Streptococcus mutans cell is Streptococcus mutans UA787 and/or Streptococcus mutans CH43. In one embodiment, mutacin I and/or III is isolated from the liquid culture after fermentation by removal of cells to obtain a cell-free supernatant. In a more specific embodiment, the fermentation broth is centrifuged to obtain a cell-free fraction, extracted with chloroform, and the emulsion layer formed between the chloroform and aqueous phases centrifuged to isolate a pellet comprising mutacin.

Other objects and advantages will become apparent from a review of the ensuing detailed description taken in conjunction with the following illustrative drawing.

### DETAILED DESCRIPTION OF THE INVENTION

Before the present method is described, it is to be understood that this invention is not limited to the particular methods, and experimental conditions described, insofar as such methods and conditions may vary. It is also to be understood that the terminology used herein is for purposes of describing particular embodiments only, and is not intended to be limiting, since the scope of the present invention will be limited only in the appended claims.

As used in this specification and in the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned are hereby incorporated by reference in their entirety herein.

Mutacin III is a class I bacteriocin (lantibiotic), produced by Streptococcus mutans UA 787. It is 22 amino acids in size, sharing striking structural similarities with epidermin (a subgroup AI lantibiotic), which is produced by Staphylococcus epidermidis. Mutacin III was found to be effective against several antibiotic-resistant pathogenic bacteria, like methicillin-resistant Staphylococcus aureus (MRSA), vancomycin-resistant Enterococcus faecium(VRE), penicillin-resistant S. pneumoniae (PRSP) (Qi et al. (1999a) Appl. Environ. Microbiol. 65:652-658). In addition, mutacin III has been found by the inventors to exert profound antimicrobial activity against Bacillus anthracis, a pathogen that may be used as an agent of bioterrorism. This finding reveals the potential of mutacin as a therapeutic agent against antibiotic-resistant pathogenic bacteria and B. anthracis.

In flask-level media screen experiments described in Example 1 below, we found that one medium can produce high-level of mutacin I/III yield, it contains yeast extract 30 g, Bacto™ peptone (DIFCO Laboratories, Detroit, Mich.) 20 g, sucrose 5 g, K₂HPO₄ 2 g, NaCl 2 g, MgSO₄φ7H₂O 1 g, distilled water, 1000 ml. Unlike on other non-producing media, both producer cells tended to form into clusters and adhered to the glass wall during incubation on this medium. To verify the production of mutacin I/III in this medium by liquid culture, further experiments were carried out in a spinner mini-bioreactor (New Brunswick Scientific Co.).

Although the genes responsible for mutacins biosynthesis are known, the mechanism for the regulation of mutacin production remains unknown (Chen et al. (1998) Appl. Environ. Microbiol. 64:2335-2340; Chen et al. (1999) Appl. Environ. Microbiol. 65:1356-1360; Qi et al. (1999a) Appl. Environ. Microbiol. 65:652-658; Woodruff et al. (1998) Gene 206:37-43). The lack of knowledge has limited the production of mutacin I/III in large scale via submerged culture.

In this study, it was observed that on the applied mutacin-producing medium, the producer cells for both mutacins clustered or clotted into pellets in both flask and Spinner Bioreactor cultivation process, whereas in all other non-producing media, both cells appeared to be isolated and dispersed thereon and no clotting or pellet formation occurred. This phenomenon appears to indicate that cell formation into a biofilm (e.g., clot and or pellet) is a prerequisite condition for mutacin I/III production in liquid culture. Thus, it may be deduced that a quorum sensing mechanism may be involved in the regulation of mutacin I/III biosynthesis.

Based on the observation of stab culture, it was presumed previously that the production of mutacin I is controlled by a cell density-mediated control mechanism (Qi et al. (2000) supra). The observation of this study further supports this presumption and suggests that mutacin III biosynthesis is regulated under the similar mechanism.

S. mutans is one of the principal bacteria responsible for dental caries (tooth decay). In the presence of sucrose, its glucosyltransferases (GTFS) enzymes enable S. mutans to produce polysaccharides (glucans and mutans) in the oral environment, which promote adherence and biofilm of cariogenic streptococci on tooth surfaces (Schilling et al. (1992) Infect. Immun. 60:284-295; Yamashita et al. (1993) Infect. Immun. 61:3811-3817). According to the observed behavior of both CH 43 and UA 787 cells in the medium used, it is expected that polysaccharides produced by both strains may also play a key role in the biofilm formation in the liquid culture applied.

The experiments below show the successful fermentation of Streptococcus mutans to produce mutacin I/III in a Spinner Bioreactor by a liquid medium. This is the first report of producing mutacin I/III in submerged culture.

Example 2 examined S. mutans UA 787 growth dynamics, mutacin III production, sucrose consumption and lactate accumulation. Exponential growth took place immediately after the inoculation and lasted until 16 h. Mutacin III biosynthesis was growth related and biomass dependent. Exponential growth took place during 4 to 8 h. The maximum cell density was achieved after 16 h and amounted to 3.888 g /L medium. Accumulation of lactic acid caused a decrease of pH, which in turn drastically inhibited the bacterial growth.

The adsorption and elution process for Mutacin III are summarized in Table 1. The whole absorption rate was 98.43%, whereas the elution rate was 94.81%. The first two elution fractions were rich with dark red to brown materials, which came from the medium. No mutacin activity was detected in these first two fractions (40% and 50 % ethanol). Mutacin III began to appear in the 60% ethanol fraction, and was centered at the fractions of 70% and 80% ethanol. The 90% fraction of ethanol only contained traces of mutacin III (3.38%).

This study demonstrates that mutacin III production was growth-related, reaching a peak at the end of the exponential growth phase. Based on the coincidence of growth and mutacin accumulation, the overproduction of mutacin could be achieved by increasing the biomass via continuous fermentation. S. mutans belongs to lactic acid bacteria (LAB), and thus obtains energy from metabolism of sugar by homofermentative fermentation, leading to the majority product, lactic acid.

In this study, accumulation of lactic acid was observed to lead to a decrease in pH, thus reducing the sucrose utilization rate. It is well known that pH is a key factor that affects the production of several bacteriocins. Nisin fermentation requires a different optimal pH for growth and lactate accumulation; thus nisin yield was improved by controlling pH (Aasen et al. (2000) Appl. Microbiol. Biotechnol. 53:159-66; Flores et al. (2001) Biotechnol. Appl. Biochem. 34(Pt. 2):103-7; Matsusaki et al. (1996) Appl. Microbiol. Biotechnol. 45:36-40). However, unlike nicin, mutacin III was mainly secreted into the fermentation broth.

### EXAMPLES

The following examples are given to provide those of ordinary skill in the art with a complete disclosure and description of how to make and use the assay, screening, and therapeutic methods of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric pressure.

### Example 1

Mutacin I and III production from Streptococcus mutans.

S. mutans CH43 was used for mutacin I production, Streptococcus mutans UA787 was used for mutacin III production. S. sanguis NY101 and Staphylococcus epidermidis strain 35984 were used as the indicator strains for the bioassay of mutacin I/III. The above four strains were grown on Todd-Hewitt (TH) plates. For mutacins fermentation, the mini-bioreactor was filled with 500 ml media and autoclaved at 121° C. for 25 min. It was inoculated with 25 ml TH 28-h cultures of CH 43 or UA787. During the fermentation process, the cultivation temperature was kept at 37° C.; the agitation rate was maintained at 150 rpm; no air was supplied to the Spinner Reactor. Samples were taken in 8-h interval for antimicrobial activity test, with the two-fold dilution method reported before (Novák et al. (1994) J. Bacteriol. 176:4316-4320; Qi et al. (1999a) Appl. Environ. Microbiol. 65:3880-3887; Qi et al. (1999b) Appl. Environ. Microbiol. 66:3221-3229). Just like their behaviors in a flask, both CH 43 and UA787 cells formed aggregates during cultivation. The highest yield of mutacin I/III achieved around at 48 h, amounting to about 16,000 AU/l and 32,000 AU/ml, respectively.

Isolation of mutacins was carried out by a modified methods described previously (Qi et al. (1999ab) supra). The broth was harvested at 72 h and centrifuged at 20,000 g for 30 min to obtain cell-free supernatant. The pooled supernatant was extracted twice with equal volumes of chloroform. The emulsion layer between chloroform and the aqueous phases was spun down; the pellet was washed twice by distilled H₂O; the remaining water-insoluble fractions were dissolved in 6M urea, and HPLC was used for further purification (Qi et al. (1999ab) supra). All purified fractions were pooled, dried in a lyophilizer, and re-dissolved in 50% acetonitrile for electrospray ionization mass spectrometry (EIMS) analysis. The EIMS showed that the molecular mass of the purified mutacin I and mutacin III obtained in this experiment were 2364 Da and 2266 Da, identical to those reported previously by the cultures of Petri dishes and PHWP membrane (Qi et al. (1999ab) supra).

### Example 2

Production of Mutacin III in a 5-liter fermentor by Streptococcus mutans UA 787

Bacterial strains and media. Streptococcus mutans UA787 was used for the mutacin III production and S. sanguis NY101 was used as the indicator for mutacin III activity assays. Both strains were stocked as frozen cultures in Todd-Hewitt broth (TH, Difco Laboratories, Detroit, Mich.) plus 15% glycerol, and subcultured on TH plates with 1.6% agar.

Fermentation. Mutacin III production was conducted in a BioFlow III fermentor (New Brunswick Scientific Co., Inc.), which contained 5-liter producing medium. The medium contained yeast extract 30 g, Bacto™ peptone (DIFCO Laboratories, Detroit, Mich.) 20 g, sucrose 5 g, K₂HPO₄ 2 g, NaCl 2 g, MgSO₄φ7H₂O 1 g, distilled water, 1000 ml. For inoculated preparation, one colony of Streptococcus mutans UA787 was inoculated into 5-ml TH broth tube from overnight culture and incubated for 12 h at 37° C. Then the whole tube culture was transferred into seed flask containing 200 ml TH broth. After a 24-h cultivation at 37° C., the seed flask was used to inoculate the BioFlow III fermentor at a rate of 4%. During the fermentation process, the cultivation temperature and agitation rate were controlled at 37° C. and 150 rpm, respectively, by AFS-Biocommand Bioprocessing Software (New Brunswick Scientific Co., Inc.); the pH was monitored by an Ingold gel pH probe (P0720-5580). No air was supplied to the fermentor during fermentation process.

Growth determination. Growth (biomass) was measured by the cell dry weight (CDW).

Samples were withdrawn aseptically from the fermentor. Aliquots (10 ml) of broth were centrifuged at 5200 g for 20 min. Centrifuged cells were washed twice by distilled water and dried to a constant weight in a vacuum oven at 80° C. The cell free supernatant was used for the following lactate and sucrose measurements.

Lactate and sucrose measurement. Lactate content in the cell-free sample supernatant was measured directly using a Sigma lactate diagnostic kit (Sigma, cat. no.735), according to the manufacturer's instructions. To measure sucrose concentration, the cell-free sample supernatant was mixed with equal an volume of 0.1 M HCl; then the mixture was heated in boiling water for 10 min to hydrolyze the sucrose into glucose and fructose. The glucose content in the hydrolysate was measured with a Sigma glucose diagnostic kit (Sigma, cat. no. 635); sucrose concentration was thus deduced by the standard curve obtained by using pure sucrose undergoing the same procedures.

Mutacin activity assay. Two different steps were used for the antimicrobial activity determination. For the measurement of cell-bound mutacin activity, centrifuged cells from sample (50 ml) was washed twice with double distilled water by centrifugation, the pellet was immersed in 2.5 ml 95% ethanol. The slurry was incubated in 37° C. for 30 min and then centrifuged. This supernatant was used to measure mutacin activity according to the plate assay method. For the measurement of secreted mutacin activity, 50 ml cell free supernatant was extracted with equal volume of chloroform, then the mixture was centrifuged and the emulsion layer was collected and lyophilized; resulted pellet was dissolved in 1 ml 6 M urea, then subjected to the plate assay method for mutacin activity assay.

Isolation of Mutacin III. Fermentation broth was harvested and centrifuged at 15,000 g at 8° C. for 25 min. The collected cell mud was washed twice with 250 ml double distilled water (pH 2.8), and then immersed in 250 ml 95% ethanol and stirred for 1 h followed by centrifugation (15,000 g for 25 min). The resulting cell mud was re-rinsed with another portion of 250 ml 95% ethanol, stirred and re-centrifuged. The ethanol solutions was combined and used for cell-bound mutacin calculation.

The cell-free broth together with combined ethanol solution obtained above were pooled and passing through a 5×22 cm column of Amberlite XAD-16 (Sigma, Amberlite XAD-16 hydrophobic polyaromatic resin, wet mesh size: 20-60), at a rate of 10 ml/min. The column was washed thoroughly with redistilled water after absorption. Elution of mutacin was carried out stepwise by 40%, 50%, 60%, 70%, 80% and 90% ethanol. Before applied for elution, each fraction was acidified to pH 2.8 by 5 M HCl. The column was regenerated by completely washing with 95% ethanol. Fractions were analyzed by HPLC with a 15 cm SOURCE 5RPC reverse-phase column, using a fragmented gradient of A (0.1% trifluoroacetic acid [TFA]) and B (0.085% TFA in 60% acetonitrile). The mutacin III concentration of each fraction was also measured by the plate assay method described previously. Fractions with mutacin activity were pooled and lyophilized to dry powder (crude powder mutacin extracts).

Purification of mutacin III by HPLC. The crude powder was dissolved by 6M urea and insoluble residues were removed by filtration. The filtrate was first applied to a reverse-phase 30 SOURCE 15RPC custom column. Elution was carried out with a fragmented gradient of solvent A and B using an AKTA Purifier (Amersham Pharmacia Biotech, Piscataway, N.J.). Active fractions were collected and lyophilized to produce pure mutacin III powder. Then it was purified again by the 15 cm SOURCE 5RPC with the same procedures described above.

TABLE 1

| | Volume | Mutacin III | Total Titer | Absorption | Elution |
|---|---|---|---|---|---|
| | (ml) | titer (AU/L) | (AU) | rate (%) | rate (%) |
| Supernatant* | 4,500 | 64,000 | 288,000 | | |
| | 4,500 | 1,000 | 4,500 | | |
| 40% ethanol | 600 | | | | |
| 50% ethanol | 600 | | | | |
| 60% ethanol | 300 | 64,000 | 19,200 | | |
| 70% ethanol | 300 | 400,000 | 120,000 | | |
| 80% ethanol | 300 | 400,000 | 120,000 | | |
| 90% ethanol | 300 | 32,000 | 9,600 | | |
| Pooled | | | 268,800 | 98.43 | 94.81 |
| titer (AU) | | | | | |

1. A method for producing a mutacin comprising:
(a) growing a mutacin-producing cell in a liquid medium under conditions in which mutacin is produced, wherein the liquid medium comprises yeast extract, peptone, sucrose, and salts; and
(b) isolating mutacin from the liquid medium.

2. The method of claim 1, wherein the liquid medium comprises 10-60 g yeast extract, 10-60 g peptone, and 10-50 g of a carbon source (weight/liter).

3. The method of claim 2, wherein the carbon source is selected from the group consisting of glucose, fructose, lactose, and sucrose.

4. The method of claim 3, wherein the liquid medium comprises 30 g yeast extract, 20 g peptone, and 20 g sucrose per liter.

5. The method of claim 1, wherein the salt content comprises K₂HPO₄, NaCl, MgSO₄.

6. The method of claim 5, wherein the salt content comprises 0.5-10 g K₂HPO₄, 1-15 g NaCl, and 0.1-20 g MgSO₄.7 H₂O.

7. The method of claim 6, wherein the liquid medium comprises 2 g K₂HPO₄, 2 g NaCl, and 1 g MgSO₄.7 H₂O per liter.

8. The method of claim 4, wherein the liquid medium comprises 30 g yeast extract, 20 g peptone, 20 g sucrose 2 g K₂HPO₄, 2 g NaCl, and 1 g MgSO₄.7 H₂O per liter.

9. The method of claim 1, wherein the mutacin produced is mutacin I and/or mutacin III.

10. The method of claim 1, wherein the mutacin-producing cell is a Streptococcus mutans cell.

11. The method of claim 10, wherein the Streptococcus mutans cell is Streptococcus mutans UA787 and/or Streptococcus mutans CH43.

12. The method of claim 1, wherein the conditions under which mutacin is produced are anaerobic liquid culture conditions under which fermentation proceeds.

13. The method of claim 12, wherein mutacin fermentation may be conducted in a bioreactor at a cultivation temperature of between 35-42° C.

14. The method of claim 13, wherein the cultivation temperature is maintained at 37° C.

15. The method of claim 12, wherein the fermentation is conducted with an agitation rate of between 50-250 rpm.

16. The method of claim 15, wherein the agitation rate is 150 rpm.

17. The method of claim 12, wherein the initial pH is between about 3.0-7.2

18. The method of claim 17, wherein the initial pH is about 5.6.

19. The method of claim 18, wherein the pH is maintained at 5.6 throughout fermentation.

20. The method of claim 1, wherein mutacin is isolated from the liquid culture after fermentation by removal of cells from the fermentation broth to obtain a cell-free liquid culture.

21. The method of claim 20, wherein the fermentation broth is centrifuged to obtain a cell-free fraction, extracted with chloroform, and the emulsion layer formed between the chloroform and aqueous phases centrifuged to isolate a pellet comprising mutacin.
